(19) United States
(12) Patent Application Publication
(10) Pub. No.: US 2009/0270270 A1
(43) Pub. Date: Oct. 29, 2009

(54) METHOD FOR DETECTING INTRAGENIC LARGE REARRANGEMENTS

(75) Inventors: Etienne Rouleau, Paris (FR); Cedrick Lefol, Andresy (FR); Sengul Tozlu-Kara, Marly Le Roi (FR); Rosette Lidereau, Gennevilliers (FR)

Correspondence Address: YOUNG & THOMPSON, 209 Madison Street, Suite 500, ALEXANDRIA, VA, 22314, US

(73) Assignee: CENTRE RENE HUGUENIN, SAINT-CLOUD (FR)

(21) Appl. No.: 12/110,535

(22) Filed: Apr. 28, 2008

(51) Int. Cl. C40B 30/04; C40B 40/06; C12Q 1/68

(52) U.S. Cl. 506/9; 506/16; 435/6

(57) ABSTRACT

The invention relates to methods for detecting at least one intragenic large rearrangement in a gene of interest, mapping the rearrangement breakpoint and diagnosing a genetic disease in a subject.

### TECHNICAL FIELD

The invention relates to a method for detecting intragenic large rearrangements and method for predicting a predisposition to a genetic disease or for diagnosing a genetic disease

### BACKGROUND OF THE INVENTION

Structural genomic alterations are recognized to play a relevant role in the pathogenesis of a growing number of diseases. Such rearrangements can range from microscopic structural alterations, including changes in quantity and structure of chromosomes, to submicroscopic small scale alterations, such as copy-number polymorphisms, microsatellites, insertions and deletions. Alterations resulting in loss or gain of genetic material may influence phenotype of a monogenic disease and its susceptibility. Even balanced rearrangements may alter expression of gene due to position effects with neighbouring the breakpoints. Most of current studies are looking for specific gene associated to a specific disease. Monogenic and oligogenic diseases are often associated with truncated mutations. Those mutations can result either a nonsense mutation, a missense mutation or a large rearrangement.

The identification of both germline and somatic genomic rearrangement is a fundamental task in molecular diagnostics since it may have important predictive, therapeutic and prognostic implications.

A wide range of molecular techniques have been developed for the detection of large genomic rearrangements. These techniques can be separated in two groups: whole-genome methods and specific loci methods.

Among the whole-genome methods, metaphase chromosome-banding techniques coupled to light microscopy were the first approach for analysing chromosomal abnormalities. Subsequently, fluorescent in situ hybridization (FISH) methods were developed for whole-genome analysis (spectral karyotyping) and recently array-based comparative genomic hybridization (array-CGH) was developed to simultaneously investigate multiple loci up to the whole genome. The whole genome approach has a low resolution to specific locus. Moreover, most patients with a medical prescription associated to a specific disease need information limited to some specific locus. Here the risk is to discover other alterations which are not in the scope of a specific disease.

Specific-loci methods are the following: Southern blotting, FISH methods designed for locus-specific analysis (metaphase, interphase and high resolution fiber FISH) and methods based on PCR amplification such as long-range PCR, quantitative/semiquantitative PCR, real-time quantitative PCR and multiplex PCR (MAPH: multiplex amplifiable probe hybridization, MLPA: multiplex ligation-dependent probe amplification, NFMP: non fluorescent multiplex PCR, QMPSF: quantitative multiplex PCR of short fluorescent fragments). Specific-loci methods, due to their high resolution, have been designed to analyse fixed positions on a gene locus that is known to be involved in pathological mechanisms, said fixed positions being generally exons. Those methods provide specific locus information. On the contrary, whole-genome methods give a panoramic view and can discover novel putative deleterious mutations. In fact, by limiting the number of points analysed, some alteration can be missed, such as alteration within an intron which is not covered by those methods. The limited number of points analysed excludes the ability to characterize the events by determining the breakpoints.

Neither specific-loci methods nor whole-genome methods are relevant methods for screening large rearrangements at a gene level (i.e. intragenic large rearrangement), as specific-loci methods are not designed for screening a region as long as current whole-genome methods do not provide a sufficient resolution and provide informations not acceptable with diagnostic methods.

Therefore, the Applicant aimed to develop a novel method designed to screen for known or putative large rearrangement in at least one gene of interest. This method is based on the analysis of the binding of a test collection and a reference collection of labelled nucleic acids with a plurality of oligonucleotides, having the following characteristics:
ranging in size from 45 to 70 nucleotides in length,
including sequences
   representative of locations distributed in the gene of interest, i.e. representative of exons and/or introns of said gene, wherein exonic regions of the gene of interest are fully covered by overlapping oligonucleotides and non-repeated intronic regions of the gene of interest are fully covered by tiling or overlapping oligonucleotides, and
   sequences representative of locations distributed in the genome outside the gene of interest,

The specific design of these oligonucleotides allows therefore the scan of the entire sequence of the exons and/or introns of the gene of interest, in order to detect precisely any known or unknown intragenic large rearrangement. The method of the invention thus provides a high-throughput technique for routine detection of intragenic large rearrangement in at least one gene of interest.

### SUMMARY OF THE INVENTION

The invention relates to a method for detecting at least one intragenic large rearrangement in at least one gene of interest, comprising:

(a) preparing a first collection of labelled nucleic acid molecules from a reference genomic source,

(b) preparing a second collection of labelled nucleic acid molecules from the genomic source to be tested,

(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
   sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
   sequences representative of locations distributed in the genome outside the gene of interest,

(d) determining the hybridization signal intensity of each oligonucleotide representative of a location distributed in the exons and/or introns of the gene of interest by subtracting
the background noise obtained by evaluating the binding of the first and the second collection of nucleic acids to said plurality of surface-bound nucleic acids representative of locations distributed in the genome outside the gene of interest, from
the binding of the first and the second collection of nucleic acids to said plurality of surface-bound nucleic acids representative of locations distributed in the exons and/or introns of the gene of interest,

thereby detecting at least one intragenic large rearrangement in said gene of interest.

In a preferred embodiment, said surface-bound nucleic acids further comprise oligonucleotides including sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

In another preferred embodiment, said surface-bound nucleic acids comprise oligonucleotides ranging in size from 55 to 60 nucleotides in length.

In another preferred embodiment, said surface-bound nucleic acids comprise oligonucleotides designed to avoid repeat sequences present in the gene of interest.

In another embodiment, said nucleic acids from first and second collection range in length from 100 to 10000 nucleotides in length.

In another embodiment, said collections of nucleic acids are distinguishably labelled and contacted with the same plurality of surface-bound nucleic acids.

Another object of the invention is a kit for detecting at least one intragenic large rearrangement in at least one gene of interest, comprising:

(a) a plurality of surface-bound nucleic acids, which comprise:
(i) oligonucleotides ranging in size from 45 to 70 nucleotides in length,
   (ii) said oligonucleotides including
   sequences representative of locations distributed in the exons and/or introns of the gene of interest, wherein exonic regions of the gene of interest are fully covered by overlapping oligonucleotides and non-repeated intronic regions of the gene of interest are filly covered by tiling oligonucleotides, and
   sequences representative of locations distributed in the genome outside the gene of interest,

(b) instructions for practicing the method according to claim 1.

In a preferred embodiment, the plurality of surface-bound nucleic acids further comprise oligonucleotides including sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

The invention provides a method for mapping an intragenic large rearrangement breakpoint in a gene of interest comprising,

(a) preparing a first collection of labelled nucleic acid molecules from a reference genomic source,

(b) preparing a second collection of labelled nucleic acid molecules from the genomic source to be tested,

(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
   sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
   sequences representative of locations distributed in the genome outside the gene of interest,

(d) determining the hybridization signal intensity of each oligonucleotide representative of a location distributed in the exons and/or introns of the gene of interest by subtracting
the background noise obtained by evaluating the binding of the first and the second collection of nucleic acids to said plurality of surface-bound nucleic acids representative of locations distributed in the genome outside the gene of interest, from
the binding of the first and the second collection of nucleic acids to said plurality of surface-bound nucleic acids representative of locations distributed in the exons and/or introns of the gene of interest,

thereby detecting at least one intragenic large rearrangement in said gene of interest,

(e) when the large rearrangement identified is a deletion, designing sens and antisens primers from the non-deleted oligonucleotides surrounding in 5′ and 3′ the sequence deleted,

(e′) when the large rearrangement identified is a duplication, designing sens and antisens primers from the duplicated oligonucleotides present in 5′ and 3′ of the sequence duplicated,

(f) carrying out a PCR with the primers designed in step (e) or (e′) on nucleic acids from reference genomic source and genomic source to be tested,

(g) sequencing the nucleic acid amplified in the PCR assay in step (f) and comparing said sequence with the known sequence of the gene of interest to map the intragenic large rearrangement breakpoint identified.

Another object of the invention is a method for predicting a predisposition of a subject to develop a genetic disease or for diagnosing a genetic disease in a subject, comprising detecting an intragenic large rearrangement in at least one gene involved in said genetic disease by

(a) preparing a first collection of labelled nucleic acid molecules from a reference genomic source,

(b) preparing a second collection of labelled nucleic acid molecules from the genomic source to be tested,

(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
   sequences representative of locations distributed in said at least one gene involved in said genetic disease, at least 100 kb before the ATG start codon for the 5′ region of said gene and at least 100 kb after the stop codon for the 3′ region of said gene, wherein exonic regions of said gene are fully covered by overlapping oligonucleotides; non-repeated intronic regions of said gene are fully covered by tiling oligonucleotides, said 5′ region of said gene is covered by at least one oligonucleotide every 250-300 bases and said 3′ region of said gene is covered by at least one oligonucleotide every 550-600 bases, and
   sequences representative of locations distributed in the genome outside the gene involved in said genetic disease,

(d) determining the hybridization signal intensity of each oligonucleotide representative of a location distributed in the exons and/or introns of the gene involved in said genetic disease by subtracting
the background noise obtained by evaluating the binding of the first and the second collection of nucleic acids to said plurality of surface-bound nucleic acids representative of locations distributed in the genome outside the gene involved in said genetic disease, from
the binding of the first and the second collection of nucleic acids to said plurality of surface-bound nucleic acids representative of locations distributed in the exons and/or introns of the gene involved in said genetic disease,

thereby detecting at least one intragenic large rearrangement in said gene involved in said genetic disease.

In a preferred embodiment, said method is a method for predicting a predisposition to cancer in a subject or for diagnosing a cancer in a subject, comprising detecting an intragenic large rearrangement in at least one gene involved in predisposition to cancer according to the method of the invention.

In a preferred embodiment, said at least one gene involved in cancer is selected from the group consisting of BRCA1, BRCA2, BRIP, PALPB1, CHEK2, PTEN, STK11, CDH1, CASP8, FGFR2, MAP3K1, ATM and TP53 genes for ovarian and breast cancers or in the group consisting of MSH2, MLH1, MSH6, MSH3, PMS1, TFGBR2, MLH3, PMS2, MYH, AXIN2 and APC genes for colorectal cancers.

In another preferred embodiment, said method is a method for predicting a predisposition to mucoviscidosis in a subject or for diagnosing mucoviscidosis in a subject, comprising detecting an intragenic large rearrangement in CFTR gene according to the method of the invention.

The invention also provides an array comprising a plurality of surface-bound oligonucleotides, wherein said oligonucleotides:
(i) range in size from 45 to 70 nucleotides,
(ii) correspond to
   sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
   sequences representative of locations distributed in the genome outside the gene of interest.

In a preferred embodiment, said array further comprises oligonucleotides including sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

In a preferred embodiment, said at least one gene of interest is selected in the group consisting of BRCA1, BRCA2, for detecting a predisposition to ovarian and breast cancer, in the group consisting of MSH6, PMS2, MSH2 and MHL1 for detecting a predisposition to colorectal cancer, and said at least one gene of interest is CFTR for detecting a predisposition to mucoviscidosis.

The invention also provides a method for validating the detection of an intragenic large rearrangement in a gene of interest, comprising

(a) when the intragenic large rearrangement identified is a deletion, designing sens and antisens primers from the non-deleted oligonucleotides surrounding in 5′ and 3′ the sequence deleted,

(a′) when the intragenic large rearrangement identified is a duplication, designing sens and antisens primers from the duplicated oligonucleotides present in 5′ and 3′ of the sequence duplicated,

(b) carrying out a quantitative or semi-quantitative PCR with the primers designed in step (a) or (a′) on nucleic acids from reference genomic source and genomic source to be tested,

(c) determining an amplification of nucleic acids from genomic source to be tested, thereby validating the presence of an intragenic large rearrangement in the gene of interest.

### BRIEF DESCRIPTION OF THE DRAWINGS

FIG. 1(A) Analysis of 5 large deletions and 2 large duplications in the BRCA1 gene. Normalized log 2 ratio for each oligonucleotide with the mean average and standard deviation of the 10 control samples without any large rearrangements in BRCA1 gene. (B) PCR product for the exon 8-13 deletion using primers based on oligonucleotide array-CGH. F1A and F1B are two patients from the same family and F2 a patient from another family. Controls 1 and 2 are two DNA samples with no known large rearrangement. (C) Sequencing showed the same breakpoint in the three patients, for a 23.8 kb deletion—intron 7 (till 38 507 324) and intron 13 (till 38 483 560).

FIG. 2(A) Analysis of a large deletion (5′ region to exon 7) in MSH2 gene (1031 oligonucleotides). (B) Analysis of a large duplication in BRCA2 (exons 17 to 20, 2749 oligonucleotides). (C) Analysis of a large duplication in MLH1 gene (exon 4, 789 oligonucleotides). (D) PCR product and breaking point determination for the large duplication in MLH1 gene (1.664 pb).

FIG. 3(A) Analysis of the exon 8-13 deletion by using oligonucleotides in exons, introns and flanking regions of BRCA1 gene—array with 3107 oligonucleotides designed for BRCA1 locus (B) Analysis of the exon 8-13 deletion by using oligonucleotides in introns and flanking region—all oligonucleotides in exons were excluded.

### DEFINITIONS

The term “intragenic large rearrangement” as used herein refers to deletion and duplication events that can be observed in a gene sequence, said sequence comprising in a restricted view introns and exons; and in an extended view introns, exons, the 5′ region of said gene and the 3′ region of said gene. The intragenic large rearrangement can also cover any gain or loss of genomic material with a consequence in the expression of the gene of interest.

The term “locus” as used herein refers to a fixed position on a chromosome.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids that are C-glycosides of a purine or pyrimidine base, polypeptides (proteins) or polysaccharides (starches, or polysugars), as well as other chemical entities that contain repeating units of like chemical structure.

The term “nucleic acid” as used herein means a polymer composed of nucleotides, e. g., deoxyribonucleotides or ribonucleotides, or compounds produced synthetically such as PNA which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g. wherein one or more of the hydroxyl groups are replaced with halogen atoms or aliphatic groups, or are functionalized as ethers, amines, or the like.

The phrase “oligonucleotide bound to a surface of a solid support” refers to an oligonucleotide or mimetic thereof such as PNA, that is immobilized on a surface of a solid substrate in a spot, where the substrate can have a variety of configurations, e.g. a sheet, bead, or other structure. In certain embodiments, the collections of features of oligonucleotides employed herein are present on a surface of the same planar support, e.g. in the form of an array.

The term “array” encompasses the term “microarray” and refers to an ordered array presented for binding to nucleic acids and the like. Arrays, as described in greater detail below, are generally made up of a plurality of distinct or different oligonucleotides bound to a surface of a solid support, also referred to as substrate immobilized nucleic acids.

An “array” includes any one-dimensional, two-dimensional or substantially two-dimensional (as well as a three-dimensional) arrangement of addressable regions bearing nucleic acids, particularly oligonucleotides or synthetic mimetics thereof and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots. A typical array may contain one or more, including more than two, more than ten, more than one hundred, more than one thousand, more ten thousand features, or even more than one hundred thousand nucleic acids, in an area of less than 20 cm² or even less than 10 cm², e. g., less than about 5 cm², including less than about 1 cm², less than about 1 mm², e.g. 100 μ², or even smaller. For example, spots may have widths (that is, diameter, for a round spot) in the range from a 10 μm to 1 cm. In other embodiments each spot may have a width in the range of 1 μm to 1 mm, usually 5 μm to 500 μm, and more usually 10 μm to 200 μm. Non-round spots may have area ranges equivalent to that of circular spots with the foregoing width (diameter) ranges. At least some, or all, of the spots are of different compositions. Inter-spots areas will typically (but not essentially) be present and will not carry any nucleic acids (or other biopolymer or chemical moiety of a type of which the spots are composed). Such inter-spots areas typically will be present where the arrays are formed by processes involving drop deposition of reagents but may not be present when, for example, photolithographic array fabrication processes are used. It will be appreciated though, that the inter-spots areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region.

For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. Arrays can be fabricated using drop deposition from pulse-jets of either nucleic acid precursor units (such as monomers) in the case of in situ fabrication, or the previously obtained nucleic acid. Also, instead of drop deposition methods, photolithographic array fabrication methods may be used.

In certain embodiments of particular interest, in situ prepared arrays are employed. In situ prepared oligonucleotide arrays, e.g. nucleic acid arrays, may be characterized by having surface properties of the substrate that differ significantly between the spots and inter-spots areas. Specifically, such arrays may have high surface energy, hydrophilic features and hydrophobic, low surface energy hydrophobic inter-spots regions. Whether a given region of a substrate has a high or low surface energy can be readily determined by determining the regions “contact angle” with water. Other features of in situ prepared arrays that make such array formats of particular interest in certain embodiments of the present invention include, but are not limited to: spot density, oligonucleotide density within each spot, spot uniformity, low intra-spot background, low inter-spot background, e.g., due to hydrophobic inter-spot regions, fidelity of oligonucleotide elements making up the individual spots, array/spot reproducibility, and the like. The above benefits of in situ produced arrays assist in maintaining adequate sensitivity while operating under stringency conditions required to accommodate highly complex samples.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first spot of interest, and the last spot of interest, even if there exist intervening areas that lack spots of interest.

An “array layout” refers to one or more characteristics of the features, such as feature positioning on the substrate, one or more feature dimensions, and an indication of a moiety at a given location.

“Hybridizing” and “binding”, with respect to nucleic acids, are used interchangeably.

The term “stringent assay conditions” as used herein refers to conditions that are compatible to produce binding pairs of nucleic acids, e.g. surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g. as in array, Southern or Northern hybridizations) are sequence dependent, and are different under different experimental parameters. Stringent hybridization conditions that can be used to identify nucleic acids within the scope of the invention can include for example hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1M NaCl, and 1% SDS at 37° C., and a wash in 1×SSC at 45° C. Alternatively, hybridization to filter-bound DNA in 0.5 MNaHP04, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include for example a salt concentration of about 0.02 molar at pH 7 and a temperature of at least about 50° C. or about 55° C. to about 60° C.; or a salt concentration of about 0.15 M NaCl at 72° C. for about 15 minutes; or a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be for example 0.2×SSC/0.1% SDS at 42° C.

A specific example of stringent assay conditions is rotating hybridization at 65° C. in a salt based hybridization buffer with a total monovalent cation concentration of 1.5 M followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Sensitivity is a term used to refer to the ability of an assay to detect the nucleic acid of interest in a sample. For example, an assay has high sensitivity if it can detect a small concentration of the nucleic acid of interest in sample. Conversely, a given assay has low sensitivity if it only detects a large concentration of the nucleic acid of interest in sample. A given assay's sensitivity is dependent on a number of parameters, including specificity of the reagents employed (such as types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labelling system, the nature of the detection system, etc.

### DETAILED DESCRIPTION OF THE INVENTION

The invention relates to a method for detecting at least one intragenic large rearrangement in at least one gene of interest comprising:

(a) preparing a first collection of labelled nucleic acid molecules from a reference genomic source,

(b) preparing a second collection of labelled nucleic acid molecules from the genomic source to be tested,

(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
   sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
   sequences representative of locations distributed in the genome outside the gene of interest,

(d) determining the hybridization signal intensity of each oligonucleotide representative of a location distributed in the exons and/or introns of the gene of interest by subtracting
the background noise obtained by evaluating the binding of the first and the second collection of nucleic acids to said plurality of surface-bound nucleic acids representative of locations distributed in the genome outside the gene of interest, from
the binding of the first and the second collection of nucleic acids to said plurality of surface-bound nucleic acids representative of locations distributed in the exons and/or introns of the gene of interest,

thereby detecting at least one intragenic large rearrangement in said gene of interest.

In one preferred embodiment of the invention, said plurality of surface-bound nucleic acids are in the form of an array or substrate immobilized oligonucleotides.

In one embodiment of the method described here above, the collections of nucleic acid may be labelled with the same label or different labels, depending on the assay protocol employed. For example, when each collection is to be contacted with different but identical arrays, each nucleic acid collection may be labelled with the same label. Alternatively, when both collections are to be simultaneously contacted with a single array of immobilized oligonucleotides, i.e. cohybridized to the same array of immobilized oligonucleotides, solution-phase collections of nucleic acids that are to be compared are generally distinguishably or differentially labelled with respect to each other.

In one embodiment of the invention, the two or more (as in certain embodiments of the invention the number of collections to be compared may be three, four or more) collections of nucleic acids are prepared from different genomic source.

The term “genome” refers to all nucleic acid sequences (coding and non-coding) and elements present in or originating from any virus, single cell (prokaryote and eukaryote) or each cell type and their organelles such as mitochondria in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulator elements needed to produce and maintain each particle, cell or cell type in a given organism.

By “genomic source” is meant the initial nucleic acids that are used as the original nucleic acid source from which the solution phase nucleic acids are produced.

The genomic source may be prepared using any convenient protocol. For example, the genomic source is prepared by first obtaining a starting composition of genomic DNA, i.e. a nuclear fraction of a cell lysate. In general, the genomic source is genomic DNA representing the entire genome from a particular organism, tissue or cell type.

In a preferred embodiment, the genomic source is mammalian and in a more preferred embodiment human.

As used herein, a “genomic source to be tested” corresponds to a genomic source where an intragenic large rearrangement is to be searched in a gene of interest and detected if present. A “reference genomic source” corresponds to a genomic source from the same organism which does not present an intragenic large rearrangement in the gene of interest. For example, a genomic source to be tested can be blood DNA from patients to be tested and the reference genomic source can be male and female DNA reference or from a pool of male and female DNA without any intragenic large rearrangement.

According to the invention, a genomic source can be provided by cells, bodily fluids or tissues. Known methods can be used to obtain a bodily fluid such as blood, sweat, tears, lymph, urine, saliva, semen, cerebrospinal fluid, feces or amniotic fluid. Similarly known biopsy methods can be used to obtain cells or tissues such as buccal swab, mouthwash, surgical removal, biopsy aspiration or the like. A genomic source can also be obtained from one or more cell or tissue in primary culture, in a propagated cell line, a fixed archival sample, forensic sample or archeological sample. Exemplary cell types from which a genomic source can be obtained include, without limitation, a blood cell such as a B lymphocyte, T lymphocyte, leukocyte, macrophage, or neutrophil; a muscle cell such as a skeletal cell, smooth muscle cell or cardiac muscle cell; germ cell such as a sperm or egg; epithelial cell; connective tissue cell such as an adipocyte, fibroblast or osteoblast; neuron; astrocyte; stromal cell; kidney cell; pancreatic cell; liver cell; or keratinocyte. A cell from which a genomic source can be obtained for use in the invention can be anormal cell or a cell displaying one or more symptom of a particular disease or condition. Thus, a genomic source used in the method of the invention can be obtained from a cancer cell, neoplastic cell, necrotic cell or the like.

In another embodiment, the genomic source may be fragmented to produce a fragmented genomic source, where the molecules have a desired average size range, such as 100 to 10000 nucleotides in length, preferably 100 to 1000 nucleotides in length. Such fragmentation may be achieved using any convenient protocol, including, but not limited to, mechanical protocols such as sonication, shearing . . . , chemical protocols such as cleavage by tris(3-hydroxy-1,2,3-benzotriazine-4(3H)one]iron(III) and other chemical agents, and enzymatic protocols such as digestion by a restriction enzyme or the like.

In another embodiment, the genomic source may be amplified with random primers in an amplifying primer extension protocol. This amplification step can be carried out prior or not to the fragmentation step.

In the method of the invention as described here above, the collections of nucleic acids are labelled with a detectable label. A number of different nucleic acid labelling protocols are known in the art and may be employed to produce a population of labelled nucleic acids. Those protocols may use for example labelled primers, labelled nucleotides, or modified nucleotides that can be conjugated with different dyes.

Primer extension reactions for generating labelled nucleic acids are well known for those skilled in the art: the primers are contacted with the template, i.e. the genomic source, and a DNA polymerase under condition sufficient to extend the primers and produce primer extension products, either in an amplifying or non-amplifying manner. DNA polymerases are for example polymerases derived from E. coli, thermophilic bacteria, archaebacteria, phage, yeasts, primates . . . . The DNA polymerase extends the primers according to the genomic template to which it is hybridized in the presence of additional reagents such as dNTPs, monovalent and divalent cations (KCl, MgCl₂), sulfhydryl reagents (DTT) and buffering agents (Tris-Hcl). The reagents employed in said primer extension reactions typically include a labelling reagent such as the primer or a labelled nucleotide, which may be labelled with a directly or indirectly detectable label. For example, in a preferred embodiment, such labelled reagent is a fluorescent tagged nucleotide such as dCTP. Fluorescent moieties that can be used to tag nucleotides include, but are not limited to, cyanine dyes such Cy-3 and Cy-5 (Amersham Inc., Piscataway, N.J.), Quasar 570 and Quasar 670 (Biosearch Technology), Alexafiluor555 and Alexafluor647 (Molecular Probes), BODIPY V-1002 and BODIPY V 1005 (Molecular Probes), POPO-3 and TOTO-3 (Molecular Probes), fluorescein and Texas red (Dupont) and POPRO3 TOPRO3 (Molecular Probes).

In the primer extension reactions employed in the method of the invention, the genomic template is typically first subjected to strand disassociation conditions, e.g. subjected to a temperature ranging from about 80° C. to about 100° C., usually from about 90° C. to about 95° C. for a period of time, and the resultant disassociated template molecules are then contacted with the primer molecules under annealing conditions, where the temperature of the template and primer composition is reduced to an annealing temperature of from about 20° C. to about 80° C., usually from about 37° C. to about 65° C.

The resultant annealed primer/template hybrids are then maintained in a reaction mixture that includes the above-discussed reagents at a sufficient temperature and for a sufficient period of time to produce the desired labelled nucleic acids. Typically, this incubation temperature ranges from about 20° C. to about 75° C., usually from about 37° C. to about 65° C. The incubation time typically ranges from about 5 min to about 18 hr, usually from about 1 hr to about 12 hr.

In a preferred embodiment of nucleic acids labelling, the nucleic acids fragments to be labelled are end-labelled to provide a collection of nucleic acids having a terminal label. The terminal label is situated distal to the substrate surface when the nucleic acids fragments are hybridized to the surface-bound oligonucleotides. The terminus of the nucleic acid fragments to be labelled is determined by the orientation of the surface-bound oligonucleotides present in the array to be used: the end that is labelled is the same that the end that anchors the surface-bound oligonucleotide to the array. Methods for end-labelling a nucleic acid fragments are well-known in the art.

Using the above protocols, at least a first collection of nucleic acids and a second collection of nucleic acids are produced from two different genomic sources, e.g. a reference genomic source and a genomic source to be tested.

In the next step of the method of the invention, the collections of labelled nucleic acids produced as described here above are contacted to a plurality of surface-bound nucleic acids under conditions such that nucleic acid hybridization to the surface-bound nucleic acids can occur. The collections can be contacted to the surface-bound nucleic acids either simultaneously or serially. In a preferred embodiment, the collections of labelled nucleic acids are contacted simultaneously with an array of distinct oligonucleotides of different sequence.

A characteristic of the invention is that the surface-bound nucleic acids that make up the spots of the arrays employed in the method are oligonucleotides having a length ranging from 45 to 70 nucleotides, preferably 55 to 65 and more preferably being 60 nucleotides long.

Another characteristic of the invention is that these surface-bound oligonucleotides have sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides.

The Applicant indeed observed that the use of overlapping oligonucleotides fully covering the exonic region allows the reinforcement of the signal intensity. In an alternative of this embodiment, exonic regions of the gene of interest may also be fully covered by tiling oligonucleotides, i.e. oligonucleotides that are not overlapping.

In another embodiment of the invention, non-repeated intronic regions of the gene of interest are fully covered by overlapping oligonucleotides.

In the meaning of the invention, overlapping oligonucleotides correspond to oligonucleotides that overlap on 10 to 35 nucleotides, preferably on 20 to 30 nucleotides, and most preferably on 30 nucleotides. For example, in a preferred embodiment of the invention, the first oligonucleotide corresponds to nucleotides 1-60 of the gene of interest, the second nucleotide corresponds to nucleotides 31-90 of the gene of interest, the third oligonucleotide corresponds to nucleotides 61-120 of the gene of interest and so on.

In the meaning of the invention, tiling oligonucleotides correspond to contiguous nucleotides. For example, in a preferred embodiment of the invention, the first oligonucleotide corresponds to nucleotides 1-60 of the gene of interest, the second nucleotide corresponds to nucleotides 61-120 of the gene of interest, the third oligonucleotide corresponds to nucleotides 121-180 of the gene of interest and so on.

Therefore, in one embodiment of the invention, the surface-bound nucleic acids that make up the spots of the arrays employed in the method are overlapping oligonucleotides that fully cover the exonic region, thereby allowing the detection of at least one intragenic large rearrangement involving the deletion or the duplication of an exonic region.

In another embodiment of the invention, the surface-bound nucleic acids that make up the spots of the arrays employed in the method are tiling or overlapping oligonucleotides that fully cover the non-repeated intronic region, thereby allowing the detection of at least one intragenic large rearrangement involving the deletion or the duplication of an intronic region.

In still another embodiment of the invention, the surface-bound nucleic acids that make up the spots of the arrays employed in the method are overlapping oligonucleotides that fully cover the exonic region and tiling or overlapping oligonucleotides that fully cover the non-repeated intronic region, thereby allowing the detection of at least one intragenic large rearrangement involving the deletion or the duplication of a region of the gene of interest.

In a more preferred embodiment of the invention, the surface-bound nucleic acids that make up the spots of the arrays employed in the method further comprise oligonucleotides having sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

In another preferred embodiment of the invention, these surface-bound oligonucleotides further have sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 100 bases and said 3′ region of the gene of interest is covered by at least one oligonucleotide every 100 bases.

In a more preferred embodiment, these surface-bound oligonucleotides further have sequences representative of locations distributed at least 100 kb before the ATG start codon for the 5′ region of the gene of interest and at least 100 kb after the stop codon for the 3′ region of the gene of interest, wherein said 5′ region of the gene of interest is covered by tiling oligonucleotides and said 3′ region of the gene of interest is covered by tiling oligonucleotides.

In a preferred embodiment of the invention, said surface-bound oligonucleotides are designed to avoid repeat sequences present in the gene of interest. These oligonucleotide sequences may for example be designed by using RepeatMasker® software.

In another preferred embodiment, said surface-bound oligonucleotides are designed to match a specific annealing temperature which is about 70-75° C. These oligonucleotide sequences may for example be designed by using specific algorithms known in the art. The size of oligonucleotides can be reduced to decrease an annealing temperature higher than 75° C.

In one embodiment of the invention, the surface-bound oligonucleotides are immobilized on a solid support such as a membrane, a glass, a plastic or a bead. The desired component may be covalently bound or non covalently attached through non specific binding, adsorption, physisorption or chemisorption. A wide variety of organic and inorganic polymers, as well as other materials, both natural and synthetic, may be employed as the material for the solid surface. Illustrative solid surfaces include nitrocellulose, nylon, glass, fused silica, diazotized membranes (paper or nylon), silicones, cellulose, and cellulose acetate. In addition, plastics such as polyethylene, polypropylene, polystyrene, and the like can be used. Other materials that may be employed include paper, ceramics, metals, metalloids, semiconductive materials, cermets or the like. In addition substances that form gels can be used. Such materials include proteins such as gelatins, lipopolysaccharides, silicates, agarose and polyacrylamides. Where the solid surface is porous, various pore sizes may be employed depending upon the nature of the system. Arrays can be fabricated using different protocols known in the art, such as drop deposition methods, or photolithographic methods.

In the next step of the method of the invention, collections of labelled nucleic acids and array of oligonucleotides are contacted to hybridize. Generally, nucleic acid hybridizations comprise the following major steps:

(1) provision of array of surface-bound nucleic acids;

(2) optionally pre-hybridization treatment to increase accessibility of surface-bound nucleic acids, and to reduce nonspecific binding;

(3) hybridization of the collections of nucleic acids to the surface-bound nucleic acids, typically under high stringency conditions;

(4) post-hybridization washes to remove nucleic acid fragments not bound in the hybridization; and

(5) detection of the hybridized nucleic acid fragments.

The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “highly stringent hybridization conditions” as used herein refers to conditions that are compatible to produce nucleic acid binding complexes on an array surface between complementary binding members, i.e. between surface-bound nucleic acids and complementary solution phase nucleic acids in a sample. Representative high stringency assay conditions that may be employed in these embodiments are provided above.

The above hybridization step may include agitation of the surface-bound nucleic acids and the sample of solution phase nucleic acids, where the agitation may be accomplished using any convenient protocol, such as shaking, rotating, spinning, and the like.

Following hybridization, the surface of surface-bound nucleic acids is typically washed to remove unbound nucleic acids. Washing may be performed using any convenient washing protocol, where the washing conditions are typically stringent, as described above.

Following hybridization and washing, as described above, the hybridization of the labelled nucleic acids to the array is then detected using standard techniques. Reading of the resultant hybridized array may be accomplished by illuminating the array and reading the location and intensity of resulting fluorescence at each spot of the array to detect any binding complexes on the surface of the array. A scanner may be used for this purpose such as for example the Agilent Microarray Scanner available from Agilent Technologies, Palo Alto, Calif. Arrays may also be read by any other reading method or apparatus known in the art such as optical techniques (for example, detecting chemiluminescent or electroluminescent labels) or electrical techniques (where each spot is provided with an electrode to detect hybridization). In the case of indirect labelling, subsequent treatment of the array with the appropriate reagents may be employed to enable reading of the array. Some methods of detection, such as surface plasmon resonance, do not require any labelling of the nucleic acids.

Results from the reading or evaluating are processed results obtained by subtracting a background measurement. The background noise is obtained by evaluating the binding of the first and the second collection of nucleic acids to said plurality of surface-bound nucleic acids representative of locations distributed in the genome outside the gene of interest.

In the meaning of the invention, “outside of the gene of interest” refers to locations present in genome areas without putative rearrangement.

Comparison of the signal intensities for a specific surface-bound oligonucleotide permits a direct comparison of copy number for a given sequence. In the method of the invention, the hybridization signal intensity between the two collections of nucleic acids and array of oligonucleotides is measured and the ratio test signal intensity versus reference signal intensity is calculated. “Test signal intensity” corresponds to the hybridization signal intensity between the second collection of nucleic acids from the genomic source to be tested and the oligonucleotides array. “Reference signal intensity” corresponds to the hybridization signal intensity between the first collection of nucleic acids from the reference genomic source and the oligonucleotides array.

The ratio Test/Reference signal intensities is equal to 1 in regions where the relative DNA sequence copy number is the same in the test and reference samples, greater than 1 in regions where relative DNA sequence copy number is greater in the test sample than in the reference sample (duplication), and less than 1 in regions where relative DNA sequence copy number is lower in the test sample than in the reference sample (deletion).

According to the invention, when relative DNA sequence copy number is greater in the test sample than in the reference sample, it corresponds to a duplication in the gene of interest, and when relative DNA sequence copy number is lower in the test sample than in the reference sample, it corresponds to a deletion in the gene of interest.

Usually ratios above/below global threshold 1.25/0.75 are considered as significant increases/decreases in relative DNA sequence copy number and therefore presence of a duplication/deletion in the gene of interest according to the invention.

In a preferred embodiment, log₂ ratios are calculated from the background-corrected intensities. Thus, a log₂ ratio of Test/Reference signal intensities is equal to 0 in regions where the relative DNA sequence copy number is the same in the test and reference samples, greater than 0 in regions where relative DNA sequence copy number is greater in the test sample than in the reference sample (duplication), and lesser than 0 in regions where relative DNA sequence copy number is lower in the test sample than in the reference sample (deletion). The Applicant considers log₂ ratios above/below global threshold 0.3/−0.3 as significant increases/decreases in relative DNA sequence copy number and therefore representative of the presence of a duplication/deletion in the gene of interest according to the invention.

In a more preferred embodiment, the log₂ ratios obtained from the above experiment is normalized to reduce signal variability using a normalization process. According to this embodiment, the normalization process is used to compare a log₂ ratio of Test/Reference signal intensities with a log₂ ratio of Negative Control/Reference signal intensities. “Negative Control signal intensity” as used herein corresponds to the hybridization signal intensity between a collection of nucleic acids from a Negative Control genomic source and the oligonucleotides array. “Negative Control” as used herein corresponds to a genomic source known to not having an intragenic large rearrangement in the gene of interest. For example, when the genomic source to be tested is blood DNA from a patient to be tested, a negative control genomic source is blood DNA from a patient tested previously as not having an intragenic large rearrangement in the gene of interest.

A data base of log₂ ratios of Negative Control/Reference signal intensities for a gene of interest may therefore be constituted.

In a preferred embodiment, the T-test used in the invention allows the normalization of a log₂ ratio of Test/Reference signal intensities with at least three log₂ ratios of Negative Control/Reference signal intensities.

The normalization process formula used in the invention is the following:

Normalized log₂ ratio of Test/Reference signal intensities=(log₂ ratio of Test/Reference signal intensities−mean of at least three log₂ ratios of Negative Control/Reference signal intensities)/standard deviation of the at least three log₂ ratios of Negative Control/Reference signal intensities

When represented on a graph, normalized log₂ ratio of Test/Reference signal intensities as obtained using the above described formula allows a better detection of an intragenic large rearrangement in a gene of interest by reducing any background noise. The goal is to reduce any variability due to SNP (single nucleotide polymorphism) or CNV (copy number variation) and enhance any rare events as large rearrangements. The Applicant considers normalized log₂ ratios above/below global threshold 4/−4 as significant increases/decreases in relative DNA sequence copy number and therefore representative of the presence of a duplication/deletion in the gene of interest according to the invention.

Another object of the invention is a kit for carrying out the method of the invention, comprising:

containers, each with one or more of the various reagents used in the methods, said containers including:
at least a plurality of surface-bound nucleic acids, which comprise:
   (i) oligonucleotides ranging in size from 45 to 70 nucleotides in length,
   (ii) said oligonucleotides including
      sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
      sequences representative of locations distributed in the genome outside the gene of interest,
reagents for labelled nucleic acids production such as random primers, buffers, dATP, dTTP, dCTP, dGTP, DNA polymerase, and labelling reagents such as labelled nucleotides,

and instructions for using the kit components in the method of the invention.

In a preferred embodiment of the invention, the ID of each oligonucleotide is associated with a specific code to facilitate the data mining on any general software such as Excel®. Each ID correspond to “gene name”, a separator “_”, “gene position” as “16” for intron 6, a separator “_”, and “the first nucleotide-position in the reference gene sequence”. In this example, BC1_I6_{—}6000 is the oligonucleotide in the reference sequence of BRCA1 positioned at 6000 and within the intron 6.

In a preferred embodiment, the plurality of surface-bound nucleic acids is in the form of oligonucleotides array.

In another embodiment, said plurality of surface-bound nucleic acids further comprises oligonucleotides including sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

The invention also provides an array comprising a plurality of surface-bound oligonucleotides, wherein said oligonucleotides are as described here above.

Another object of the invention is a method for mapping an intragenic large rearrangement breakpoint in a gene of interest comprising,

(a)-(d) detecting at least one intragenic large rearrangement in said gene of interest according to the method described here above, wherein the plurality of surface-bound oligonucleotides comprises oligonucleotides ranging in size from 45 to 70 nucleotides, and having sequences representative of locations distributed in the gene of interest and locations distributed at least 100 kb before the ATG start codon for the 5′ region of the gene of interest and at least 100 kb after the stop codon for the 3′ region of the gene of interest, wherein—exonic regions of the gene of interest are fully covered by overlapping oligonucleotides,

non-repeated intronic regions of the gene of interest are fully covered by tiling or overlapping oligonucleotides, and

5′ region of the gene of interest is covered by tiling oligonucleotides and said 3′ region of the gene of interest is covered by tiling oligonucleotides.

(e) when the large rearrangement identified is a deletion, designing sens and antisens primers from the non-deleted oligonucleotides surrounding in 5′ and 3′ the sequence deleted,

(e′) when the large rearrangement identified is a duplication, designing sens and antisens primers from the duplicated oligonucleotides present in 5′ and 3′ of the sequence duplicated,

(f) carrying out a PCR with the primers designed in step (e) or (e′) on nucleic acids from reference genomic source and genomic source to be tested,

(g) sequencing the nucleic acid amplified in the PCR assay in step (f) and comparing said sequence with the known sequence of the gene of interest to map precisely the intragenic large rearrangement breakpoint identified.

As used herein, the term “breakpoint mapping” refers to the characterization of the precise molecular localisation of any intragenic large rearrangement in a gene of interest.

This method allows the design of specific primers from the oligonucleotides surrounding the breakpoint, in order to map precisely at the nucleotide scale the breakpoint.

No amplification is generally observed when the PCR is carried out on nucleic acids from reference genomic source as selected primers are located too far on the DNA sequence.

Amplification is generally observed when the PCR is carried out on nucleic acids from the genomic source to be tested as selected primers surround the sequence deleted or are present in 5′ and 3′ of the duplicated sequence. Said PCR test can be considered as a first validation test for the detection of the intragenic large rearrangement in the gene of interest.

Another object of the invention is a method for predicting a predisposition of a subject to develop a genetic disease or for diagnosing a genetic disease in a subject, comprising detecting an intragenic large rearrangement according to the method described here above in at least one gene involved in said genetic disease.

For example, detecting an intragenic large rearrangement in the following genes may allow the prediction of a predisposition of the subject to develop a corresponding disease or may allow the diagnosis of said corresponding disease in said subject.

| Genes | Related disease |
|---|---|
| ABL oncogene | Glioblastome |
| p53 gene | Soft tissue sarcoma |
| THRA1 gene | Breast cancer |
| TPM3 and NTRK1 genes | Thyroid papillary carcinoma |
| EGFR gene | Pulmonary cancer |
| WT1 gene | Wilm tumor |
| RET gene | Multiple endocrine neoplasia |
| Betacatenin gene | Endometer cancer |
| KIT gene | Gastrointestinal stroma tumor |
| RB1 gene | Retinoblastoma |
| BRCA1/BRCA2 genes | Breast and ovarian cancer |
| MSH1/MLH1 genes | Lynch syndrome |
| MECP2 gene | Rett syndrome |
| LDL receptor gene | Familial hypercholesterolemia |
| Apolipoprotein gene | Hypercholesterolemia |
| KCNH2 gene | Romano-Ward syndrome |
| Neurofibromatosis ½ genes | Von Recklinhausen neurofibromatosis |
| | NF1/2 |
| HMGA2 and NFIB genes | Lipoma |
| APC gene | Familial polyposis |
| Bcr-abl genes | Chronic lymphoid leukaemia |
| Red cell membrane glycophorin | Elliptocytosis |
| C gene | |
| ALL1 gene | Acute leukaemia |
| CDKN2 gene | Malign lymphoma |
| Protein S gene | Hereditary deficit in protein S |
| Factor VIII gene | Haemophilia A |
| CFTR | Mucoviscidosis |
| Alphagalactosidase gene | Fabry disease |
| Mucopolysaccharide genes | Hunter syndrome |
| SRD5A2 gene | Deficit in 5 alpha reductase type 2 |
| COL4A5 gene | Alport type juvenile syndrome |
| GRIK4 gene | Schizophrenia and bipolar disorder |
| Parkin gene | Parkinson disease |
| PCDH15 gene | Usher syndrome |
| PRPF31 gene | Retinitis pigmentosa |
| Dystrophin gene | Duchenne's myopathy |

Another object of the invention is a method for predicting a predisposition of a subject to develop a cancer or for diagnosing a cancer in a subject, comprising detecting an intragenic large rearrangement according to the method described here above in at least one gene involved in cancer.

In a preferred embodiment, said gene involved in cancer is selected from the group consisting of BRCA1, BRCA2, BRIP, PALPB1, CHEK2, PTEN, STK11, CDH1, CASP8, FGFR2, MAP3K1, ATM and TP53 genes for ovarian and breast cancers or in the group consisting of MSH2, MLH1, MSH6, MSH3, PMS1, TFGBR2, MLH3, PMS2, MYH, AXIN2 and APC genes for colorectal cancers.

In a more preferred embodiment, said gene involved in cancer is selected among BRCA1, BRCA2 for predicting a predisposition to or diagnosing a breast cancer.

In a more preferred embodiment, said gene involved in cancer is selected among MSH2, MSH6, PMS2 and MLH1 for predicting a predisposition to or diagnosing a colorectal cancer.

In a more preferred embodiment, said gene involved in cancer is selected among MYH/APC for predicting a predisposition to or diagnosing a familial polyposis associated to colorectal cancer.

Another object of the invention is a method for predicting a predisposition of a subject to develop mucoviscidosis or for diagnosing mucoviscidosis in a subject, comprising detecting an intragenic large rearrangement according to the method described here above in CFTR gene.

Another object of the invention is an array as described here above, wherein said gene of interest is selected among BRCA1, BRCA2, MSH2, MHL1, CFTR.

Another object of the invention is an array as described here above, designed for the detection of an intragenic large rearrangement in more than one gene of interest.

Preferably, said genes of interest are selected in the group consisting of BRCA1, BRCA2, BRIP, PALPB1, CHEK2, PTEN, STK11, CDH1, CASP8, FGFR2, MAP3K1, ATM and TP53 genes for ovarian and breast cancers or MSH2, MLH1, MSH6, MSH3, PMS1, TFGBR2, MLH3, PMS2, MYH, AXIN2 and APC genes for colorectal cancers.

In a preferred embodiment, said at least one gene of interest is selected in the group consisting of BRCA1 and BRCA2 selected for ovarian and breast cancer, in the group consisting of MSH6, PMS2, MSH2 and MHL1 for colorectal cancer.

In another preferred embodiment, the invention provides an array as described here above, wherein the gene of interest of CFTR.

The invention also provides a method for validating the detection of an intragenic large rearrangement in a gene of interest, comprising

(a) when the intragenic large rearrangement identified is a deletion, designing sens and antisens primers from the non-deleted oligonucleotides surrounding in 5′ and 3′ the sequence deleted,

(a′) when the intragenic large rearrangement identified is a duplication, designing sens and antisens primers from the duplicated oligonucleotides present in 5′ and 3′ of the sequence duplicated,

(b) carrying out a quantitative or semi-quantitative PCR with the primers designed in step (a) or (a′) on nucleic acids from reference genomic source and genomic source to be tested,

(c) determining an amplification of nucleic acids from genomic source to be tested, thereby validating the presence of an intragenic large rearrangement in the gene of interest.

Examples of quantitative or semi-quantitative PCR assay to be carried out are the following: real time quantitative PCR, quantitative multiplex PCR of short fluorescent fragment (QMPSF), or multiplex ligation-dependent probe amplification (MLPA).

### EXAMPLES

In the following description, all experiments for which no detailed protocol is given are performed according to standard protocol.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Methods

Patients

We analyzed genomic DNA from nine patients with large rearrangements in BRCA1 exons. These large rearrangements were detected by MLPA, QMPSF and/or gene dosage assay and similar large rearrangements had already been reported in these exons. The breakpoints in these patients were unknown. The rearrangements consisted of five large deletions and two large duplications. Two patients from the same family and one patient from another family had deletions affecting the same exons (exons 8 to 13). The other deletion events affected exon 3, exon 15, exon 22, and the 5′ region to exon 2. The duplication events affected exon 13 and exons 18-20.

The positive control was somatic DNA with a duplication of chromosome arm 17q, including the BRCA1 locus.

Ten negative controls were used, consisting of a pool of female DNA, a pool of male DNA, and eight samples from individuals without large rearrangements in the BRCA1 gene.

To extent the application of the method to other genes, we analyzed in another experiment four samples: one with a duplication in exons 17 to 20 of BRCA2, one with a duplication in exon 4 of MLH1 and 2 with deletions in exons of MSH2 (exons 8-10, exons 1-7). In another experiment, we also prove the ability of the method to detect large rearrangements only with oligonucleotides in intronic and flanking regions. Here, an assay was also performed with the deletion of exons 8 to 13 of BRCA1 with a higher density array.

Genomic DNA Extraction

Genomic DNA was extracted from peripheral blood lymphocytes by column extraction with the QIAmp DNA blood kit (Qiagen® Courtaboeuf, France). The quantity and quality of all experimental DNA samples were assessed with Nanodrop® technology and verified by agarose gel electrophoresis. DNA working solutions were prepared with an approximate concentration of 100 ng/μL.

Probe Design and Arrays

An 11 000-oligonucleotide microarray was specially designed with home-designed oligonucleotides and with oligonucleotides representative of locations distributed in the genome outside the gene of interest and all over the genome (from Agilent catalogue and home-design).

In the first assay, a dedicated array was design to large rearrangement in BRCA1 gene (Rouleau E, et al. Clin Genet. 2007 Sep;72(3):199-207).

8505 were located throughout the genome (1481 Agilent oligonucleotides and 7024 home-designed oligonucleotides), while 1679 were specifically home-designed and dedicated for the BRCA1 gene and its flanking regions.

The reference sequence included the BRCA1 gene and its flanking regions: 168 kb before the ATG start codon for the 5′ region, and 230 kb after the TGA stop codon for the 3′ region. In the Human Genome 17 coding system, the oligonucleotides were located from nucleotide 38 220 688 to nucleotide 38 695 827 in chromosome 17.

The oligonucleotide sequences were designed by using specific algorithms and RepeatMasker® software to avoid repeat sequences and to obtain some overlap. Most oligonucleotides were 60 nucleotides long and were chosen to match a specific annealing temperature. The interval between two oligonucleotides depended on the genome region and on the presence of repeat sequences. The exonic regions were fully covered by overlapping and duplicated probes. Table 1 shows the oligonucleotide distribution. The selection algorithm and the presence of repeat sequences explain why a few introns were hardly covered by any of the oligonucleotides.

TABLE 1
Distribution of 1679 oligonucleotides in the 5′ region, exons,
introns and 3′ region of the BRCA1 gene. Exons were extensively
covered with repeated and overlapping oligonucleotides.
Oligonucleotides covering introns and external regions respected a
specific spacing and avoided repeated or homologous sequences.

| | Number of | Number of | Average |
|---|---|---|---|
| Region | oligonucleotides | bases | spacing |
| 5′ region | 586 | 167281 | 285 |
| Exon 2 | 11 | 99 | 9 |
| Intron 2 | 7 | 8237 | 1177 |
| Exon 3 | 9 | 54 | 6 |
| Intron 3 | 12 | 9192 | 766 |
| Exon 5 | 9 | 78 | 9 |
| Intron 5 | 2 | 1499 | 750 |
| Exon 6 | 12 | 89 | 7 |
| Intron 6 | 3 | 606 | 202 |
| Exon 7 | 9 | 140 | 16 |
| Intron 7 | 5 | 4241 | 848 |
| Exon 8 | 17 | 106 | 6 |
| Intron 8 | 0 | 2485 | — |
| Exon 9 | 6 | 46 | 8 |
| Intron 9 | 1 | 1321 | 1321 |
| Exon 10 | 16 | 77 | 5 |
| Intron 10 | 2 | 985 | 493 |
| Exon 11 | 250 | 3426 | 14 |
| Intron 11 | 1 | 402 | 402 |
| Exon 12 | 17 | 89 | 5 |
| Intron 12 | 15 | 8368 | 558 |
| Exon 13 | 26 | 172 | 7 |
| Intron 13 | 18 | 5789 | 322 |
| Exon 14 | 11 | 127 | 12 |
| Intron 14 | 7 | 1966 | 281 |
| Exon 15 | 25 | 191 | 8 |
| Intron 15 | 3 | 3092 | 1031 |
| Exon 16 | 41 | 311 | 8 |
| Intron 16 | 10 | 3232 | 323 |
| Exon 17 | 9 | 88 | 10 |
| Intron 17 | 8 | 3656 | 457 |
| Exon 18 | 8 | 78 | 10 |
| Intron 18 | 10 | 500 | 50 |
| Exon 19 | 8 | 41 | 5 |
| Intron 19 | 4 | 6197 | 1549 |
| Exon 20 | 17 | 84 | 5 |
| Intron 20 | 6 | 5934 | 989 |
| Exon 21 | 22 | 55 | 3 |
| Intron 21 | 4 | 1868 | 467 |
| Exon 22 | 20 | 74 | 4 |
| Intron 22 | 1 | 1417 | 1417 |
| Exon 23 | 8 | 61 | 8 |
| Intron 23 | 1 | 1840 | 1840 |
| Exon 24 | 13 | 124 | 10 |
| 3′ region | 409 | 230470 | 563 |

In another assay, we designed several others genes and redesigned BRCA1 array. A breast and ovarian array was designed. There were 3107 oligonucleotides for BRCA1, 2749 for BRCA2, and 4339 oligonucleotides outside the genes of interest and all over the genome. A colorectal array as designed: 1045 for MLH1, 1114 for MSH2, 1099 for MSH6, 567 for MLH3, 1624 for APC, 1250 for PMS1, 466 for PMS2 and 1225 for TGFBR2. 1813 oligonucleotides were outside those genes and all over the genome.

Oligonucleotide Array-CGH

Each CGH hybridization run was performed as recommended in the Agilent manual. Genomic DNA (1,5 μg) from reference samples (46, XX female or 46, XY male) and experimental samples was digested with 5 μL of AluI (50 units) and 5 μL of RsaI (50 units) (Promega, Madison, USA) in 10 μL of buffer C (Promega, Madison, USA) in a final volume of 100 μL. All digests were run for 2 h at 37° C. Reference and experimental samples were then purified by using the QIAQuick PCR clean-up kit (Qiagen, Courtaboeuf, France). The reference samples were female and male DNA pools. Labeling reactions were performed with 21 μL (800 ng) of purified restricted DNA and 29 μL of Bioprime array CGH Genomic labeling kit solution (Invitrogen, Carlsbad, USA) according to the manufacturer's instructions, for 2 h at 37° C. The labeling kit solution was composed of 20 μL of random primer, 5 μL of 10×dUTP mix and 3 μL of Cy5dUTP or Cy3dUTP at 1 mM, and 1 μL of Exo Klenow (40 units—Invitrogen, Carlsbad, USA). Labeled targets were filtered on a Microcon YLM-30 column (Millipore, Billerica, USA) and adjusted to a final volume of 75 μL. For each hybridization run, 75 μL of labeled sample was mixed with 25 μL of human COT-1 DNA (1 mg/ml—Invitrogen, Carlsbad, USA), 25 μL of Agilent 10× blocking reagent, and 125 μL of Agilent 2× hybridization buffer (Agilent technologies, Santa Clara, USA) in a final volume of 250 μL. The experimental and reference hybridization mixes were pooled, yielding a final volume of 500 μL. Before hybridization to the array, the 500-μL mix was denatured at 95° C. for 3 minutes and held for 30 min at 37° C. The sample was dispensed onto a gasket slide on which the array was plated. The sandwiched slides were placed in the Agilent microarray hybridization (Agilent Technologies, Santa Clara, USA) chamber and hybridization was carried out for 40 h in a 65° C. oven rotating at 15 rpm.

The arrays were disassembled in a disassembly wash at room temperature, then washed for 5 min at room temperature in Agilent Oligo aCGH Wash Buffer 1 followed by 1 min at room temperature in Agilent Oligo aCGH Wash Buffer 2 (Agilent Technologies, Santa Clara, USA). The slides were dried and scanned with an Agilent G2565-AA DNA microarray scanner (Agilent Technologies, Santa Clara, USA).

Data Analysis

Microarray images were analyzed by using Feature Extraction® software (version 8.5.1.1 Agilent Technologies) at default settings. No filter was applied to the data sets. The global quality of the experiment was validated against the quality metrics of this software.

Data analysis was performed with CGH-analytics® from Agilent Technologies and with Microsoft Excel®. The sample/reference intensity ratio reflects the copy number and usually ranges from 1/2 (deletions) to 3/2 (duplications). The sample/reference intensity ratio was log 2-transformed, and the results thus fluctuated in Gaussian fashion around zero.

Qualitative analysis was used first. The panoramic view of the gene gave the precise location of the deleted or duplicated sequence. A normalization method (patient versus control samples) was used to reduce signal variability. These graphs helped to locate the large rearrangements.

We used a threshold log 2 ratio-based approach to assess the number of false-positives and false-negatives for each oligonucleotide. The threshold was computed from log 2 intensity ratios of control samples in order to reduce the number of misclassified oligonucleotides. We computed the false-positive rate by limiting the analysis to control samples and to normal areas of other samples, i.e. all areas except those containing exons and introns involved in a large rearrangement. We computed the false-negative rate by using only exons known to bear large rearrangements. We used the F-test to compare the variability and misclassification rate between the Agilent and home-designed oligonucleotides.

We did not use “moving averages” solutions to assess the size and breakpoints of large rearrangements, owing to the tiling and overlapping of the oligonucleotides. Oligonucleotides with the last in-threshold signals were chosen to define the region of duplication or deletion.

Breakpoint Determination

The breakpoints were located by using classical PCR amplification followed by sequencing. The reference sequence was the selected oligonucleotides surrounding the breakpoints. The primers were selected in this sequence with OLIGO6® (Molecular Biology—Insights) and PRIMER3®.

We systematically chose two sets of primers to nest the PCR. The deletion from exon 8 to 13 is used here as an example.

The primers were 5′-CATCAGATACACCAAAAAGACAGA-3′ (SEQ ID NO:1) and 5′-TATTTACTCCTCCAAATGTATCACT-3′ (SEQ ID NO:2), encompassing 25 862 bp in a wild-type allele. For this specific large rearrangement the thermal cycling conditions comprised one denaturation cycle at 96° C. for 10 min, 30 to 40 cycles at 96° C. for 45 s, and 55° C. for 30 s, 72° C. for 1 to 3.5 min and 72° C. for 10 min, then 4° C. for ever. The PCR run were performed in a 50-μL volume containing 100 ng of genomic DNA, 20 pmol/μL each primer, 2.5 μL of reaction buffer, 1.15 mmol/L dNTPs and 1 unit of AmpliTaq Gold (Applied Biosystem, Courtaboeuf, France). The PCR products were analyzed on agarose gel and then purified and sequenced in both directions by using each PCR primer with the BigDye Terminator Cycle Sequencing Reaction Kit (Applied Biosystem, Courtaboeuf, France) and an ABI Prism 3030 automated sequencer. The cycling conditions consisted of 25 cycles at 94° C. for 30 s, 50° C. for 15 s, and 60° C. for 2 min.

Results

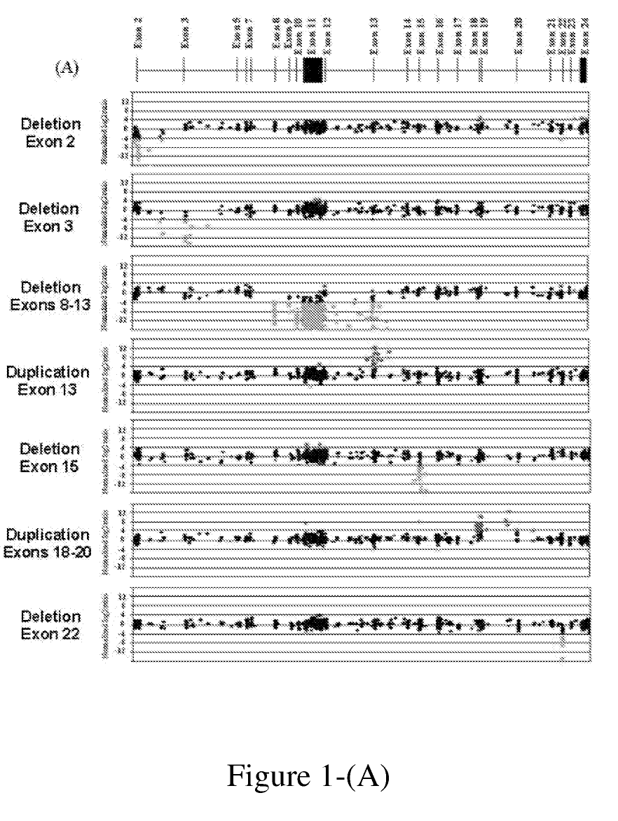

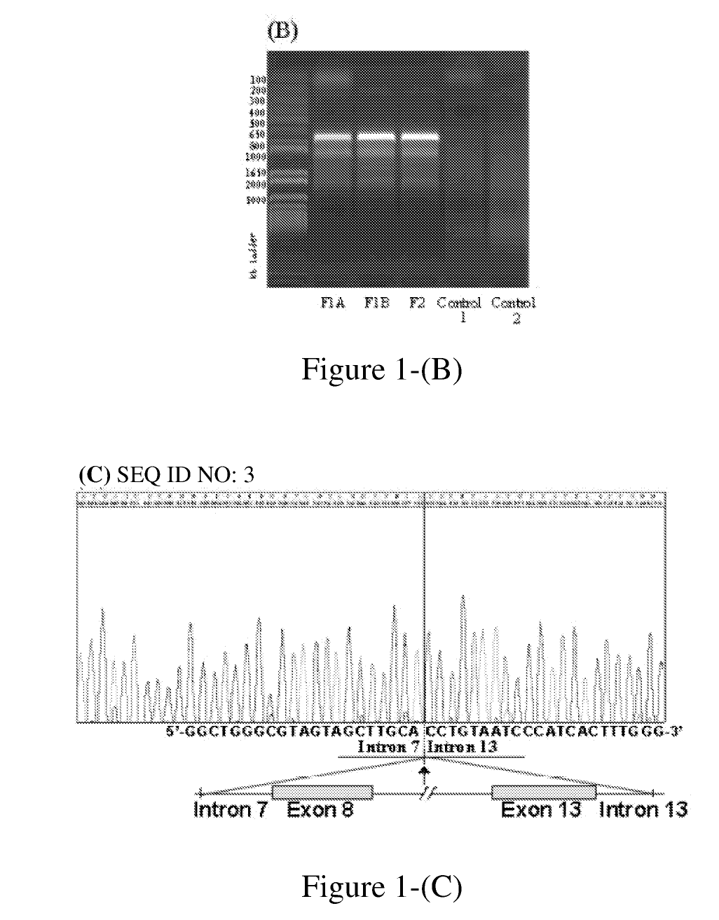

We developed a dedicated array designed to detect large rearrangements in the BRCA1 gene and its flanking regions. The resulting high-resolution oligonucleotide array-CGH approach gave a panoramic view of the gene. Nine patient DNA samples and 11 control samples were analyzed. All the oligonucleotides in the array were informative for all the samples (FIG. 1-A). The somatic DNA sample with 17q duplication bore more than two copies of the whole BRCA1 gene and its flanking regions. No large rearrangements in the BRCA1 gene were detected in the 10 control samples. All the known large rearrangements in the BRCA1 gene the 9 patient samples were successfully detected. No other deletion or duplication events were detected in the BRCA1 gene.

Quality of the Oligonucleotide Design

To assess the quality of the oligonucleotide design, the intensity ratios of the commercial oligonucleotides were compared with those of the home-designed and dedicated oligonucleotides. With the 1481 Agilent oligonucleotides, the mean log₂ intensity ratio was −0.005 and the standard deviation was 0.327 (28 139 oligonucleotides of 19 samples without somatic DNA). For the home-designed oligonucleotides, the mean log₂ intensity ratio was −0.005 and the standard deviation was 0.280 (165 357 oligonucleotides for 19 samples without somatic DNA). The standard deviation of the home-designed oligonucleotides was significantly lower than that of the Agilent oligonucleotides (F-test, P<0.001).

For threshold values of [−0.3;+0.3], we found a very similar rate of false-positives in normal samples and normal areas: 10% with Agilent oligonucleotides (for 19 samples without somatic DNA; 28 139 oligonucleotides) and 11% for BRCA1 home-designed oligonucleotides (for 10 control samples; 16 790 oligonucleotides).

For a threshold of [−0.3;+0.3], we obtained 85% of true-positives for 2870 oligonucleotides in exons bearing large rearrangements. The somatic DNA with the whole-gene deletion had a true-positive rate of 89%, while the deletion from exons 8 to 13 gave 88% of true-positives. With the exon 22 deletion, only seven of the 20 relevant oligonucleotides were positive. These results suggested a significant impact of the number and density of oligonucleotides in the areas affected by large rearrangements.

The information provided by oligonucleotide array-CGH helped us to design the best PCR primers and to amplify the variant alleles. We readily obtained PCR products smaller than 5 kb for the deletions, with the exception of the deletion from the 5′ region to exon 2 (see above).

BRCA1 Array

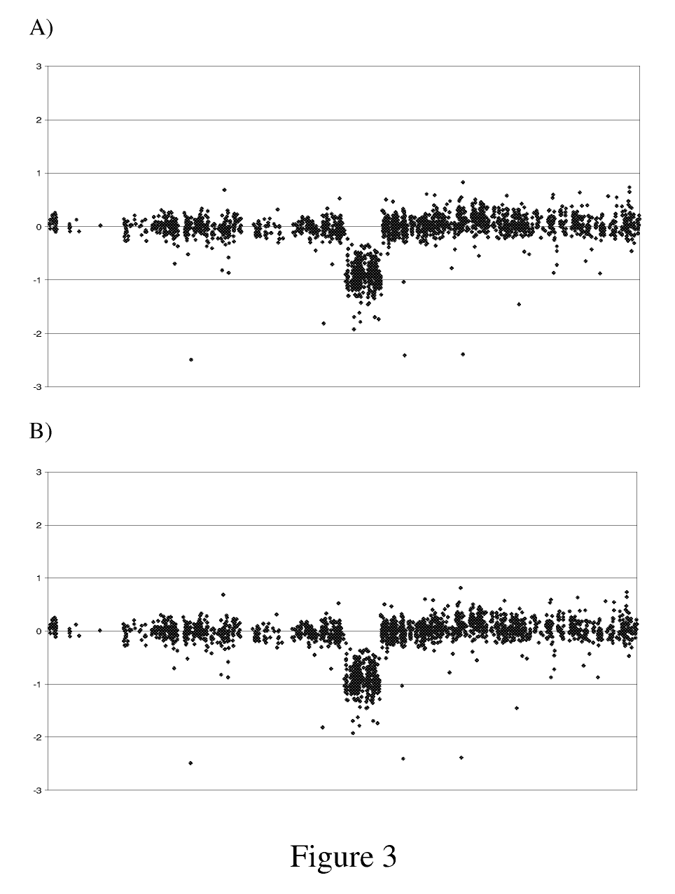

Deletion from Exon 8 to Exon 13 (FIGS. 1 and 3)

The estimated size of this deletion, based on oligonucleotide array-CGH, was between 20 and 24 kb. The size reported in the literature is 23.8 kb. We were able to sequence this region and to determine the breakpoint by using a simple PCR assay. The deleted sequence had a precise size of 23 763 bp (FIGS. 1-B and 1-C). The two families (3 patients) tested had exactly the same breakpoint. With the new BRCA1/BRCA2 design, we prove in another assay that the information obtained by excluding oligonucleotides in exons was similar to the full array on the gene of interest (see FIGS. 3A and 3B).

Deletion of Exon 3

The estimated size, based on oligonucleotide array-CGH, was between 8.3 kb and 12.2 kb. We obtained a specific PCR product and a sequence giving a precise size of 11 413 bp. In the literature, the reported size of another familial exon 3 deletion was 1039 bp.

Deletion of Exon 15

The estimated size, based on oligonucleotide array-CGH, was between 1.9 and 5.0 kb. The deleted sequence had a precise size of 2998 bp, as reported in the literature.

Deletion of Exon 22

The estimated size obtained with the oligonucleotide array-CGH was between 0.3 and 1.7 kb. The deleted sequence had a precise size of 510 bp, as reported in the literature.

Deletion of the 5′ Region to Exon 2

When the analysis was restricted to exon 2, 82% of the oligonucleotides gave true-positive results for this deletion. In the 5′ region, the percentage of true-positives ranged from 10 to 43%, owing to the high density of homologous sequences and pseudogenes. The estimated size obtained with the oligonucleotide array-CGH was 40.4 to 58.1 kb, corresponding to BRCA1 exon 2, the entire NBR2 gene, and possibly the beginning of the NBR1 gene in the 5′ region of BRCA1. A specific PCR product brought a similar result of 36.9 kb as reported in the literature.

Duplication of Exon 13

The estimated size was between 5 kb and 7.9 kb. A specific PCR product brought a similar result of 6.1 kb as reported in the literature.

Duplication from Exon 18 to 20

The estimated size was between 7.4 kb and 10.9 kbA specific PCR product brought a similar result of 8.7 kb as reported in the literature.

Except for the deletion of the 5′ region, the size estimated with the oligonucleotide array-CGH approach was around 1-2 kb to the real size, obtained by PCR or reported in the literature. With high-resolution oligonucleotide array-CGH, we were able to characterize the breakpoint and to obtain, in most cases, a specific PCR product.

BRCA1, BRCA2, MLH1, MSH2 Arrays

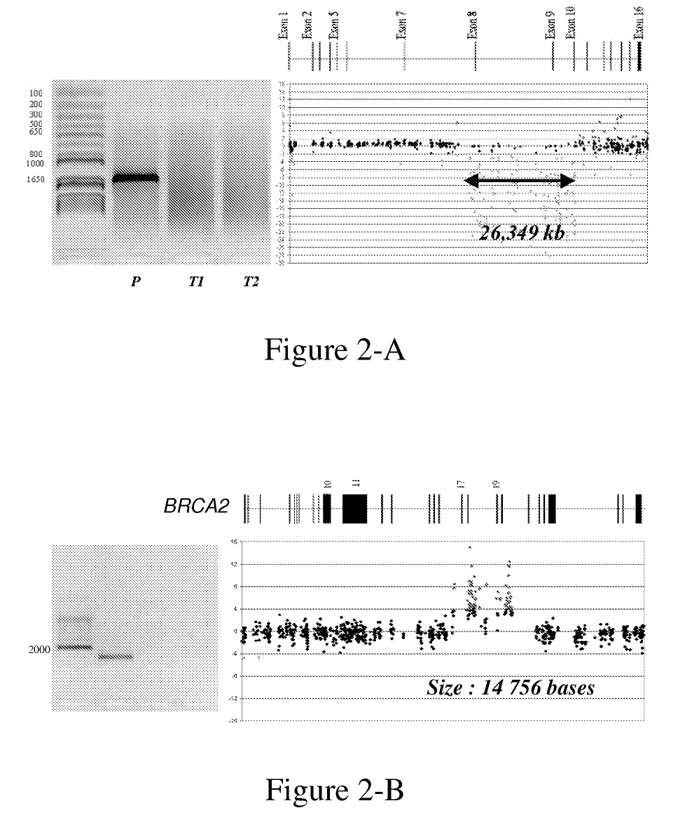

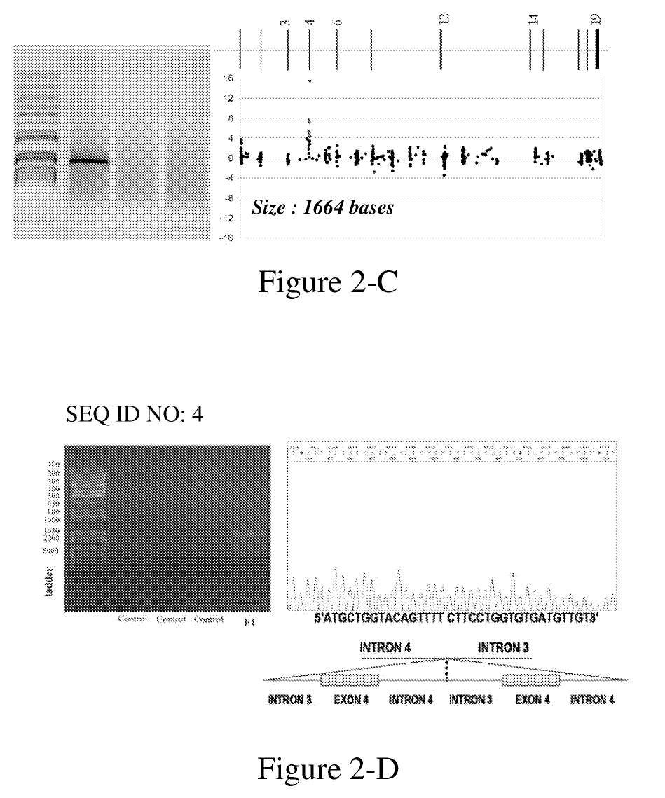

Deletion from Exons 8 à 10 of MSH2 Gene (FIG. 2A)

The estimated size was 23.6 to 26.3 kb. A specific PCR product brought a precise size of 26 349 bases, never reported in the literature.

Duplication from Exons 17 to 20 of BRCA2 Gene (FIG. 2B)

The estimated size was 8.3 to 12 kb. A specific PCR product brought a precise size of 14 756 bases, never reported in the literature.

Duplication of Exon 4 of MLH1 Gene (FIGS. 2C and D)

The estimated size was 789 oligonucleotides. A specific PCR product brought a precise size of 1664 bases, never reported in the literature.

1. A method for detecting at least one intragenic large rearrangement in at least one gene of interest, comprising:
(a) preparing a first collection of labelled nucleic acid molecules from a reference genomic source,
(b) preparing a second collection of labelled nucleic acid molecules from the genomic source to be tested,
(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
sequences representative of locations distributed in the genome outside the gene of interest,
(d) determining the hybridization signal intensity of each oligonucleotide representative of a location distributed in the exons and/or introns of the gene of interest by subtracting
the background noise obtained by evaluating the binding of the first and the second collection of nucleic acids to said plurality of surface-bound nucleic acids representative of locations distributed in the genome outside the gene of interest, from
the binding of the first and the second collection of nucleic acids to said plurality of surface-bound nucleic acids representative of locations distributed in the exons and/or introns of the gene of interest,
thereby detecting at least one intragenic large rearrangement in said gene of interest.

2. The method according to claim 1, wherein said surface-bound nucleic acids further comprise oligonucleotides including sequences representative of locations distributed at least 100 kb before the ATG start codon for the 5′ region of the gene of interest and at least 100 kb after the TGA stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

3. The method according to claim 1, wherein said surface-bound nucleic acids comprise oligonucleotides ranging in size from 55 to 60 nucleotides in length.

4. The method according to claim 1, wherein said surface-bound nucleic acids comprise oligonucleotides designed to avoid repeat sequences present in the gene of interest.

5. The method according to claim 1, wherein said nucleic acids from first and second collection range in length from 100 to 10000 nucleotides in length.

6. The method according to claim 1, wherein said collections of nucleic acids are distinguishably labelled and contacted with the same plurality of surface-bound nucleic acids.

7. A kit for detecting at least one intragenic large rearrangement in at least one gene of interest, comprising:
(a) a plurality of surface-bound nucleic acids, which comprise:
(i) oligonucleotides ranging in size from 45 to 70 nucleotides in length,
(ii) said oligonucleotides including
sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
sequences representative of locations distributed in the genome outside the gene of interest,
(b) instructions for practicing the method according to claim 1.

8. The kit according to claim 7, wherein the plurality of surface-bound nucleic acids further comprise oligonucleotides including sequences representative of locations distributed at least 100 kb before the ATG start codon for the 5′ region of the gene of interest and at least 100 kb after the TGA stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

9. A method for mapping an intragenic large rearrangement breakpoint in a gene of interest comprising,
(a) preparing a first collection of labelled nucleic acid molecules from a reference genomic source,
(b) preparing a second collection of labelled nucleic acid molecules from the genomic source to be tested,
(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
sequences representative of locations distributed in said gene of interest, and locations distributed at least 100 kb before the ATG start codon for the 5′ region of said gene and at least 100 kb after the stop codon for the 3′ region of said gene, wherein exonic regions of said gene are fully covered by overlapping oligonucleotides, non-repeated intronic regions of said gene are fully covered by tiling oligonucleotides, and 5′ region and 3′ region of said gene are fully covered by tiling oligonucleotides, and
sequences representative of locations distributed in the genome outside the gene of interest,
(d) determining the hybridization signal intensity of each oligonucleotide representative of a location distributed in the exons and/or introns of the gene of interest by subtracting
the background noise obtained by evaluating the binding of the first and the second collection of nucleic acids to said plurality of surface-bound nucleic acids representative of locations distributed in the genome outside the gene of interest, from
the binding of the first and the second collection of nucleic acids to said plurality of surface-bound nucleic acids representative of locations distributed in the exons and/or introns of the gene of interest,
thereby detecting at least one intragenic large rearrangement in said gene of interest,
(e) when the large rearrangement identified is a deletion, designing sens and antisens primers from the non-deleted oligonucleotides surrounding in 5′ and 3′ the sequence deleted,
(e′) when the large rearrangement identified is a duplication, designing sens and antisens primers from the duplicated oligonucleotides present in 5′ and 3′ of the sequence duplicated,
(f) carrying out a PCR with the primers designed in step (e) or (e′) on nucleic acids from reference genomic source and genomic source to be tested,
(g) sequencing the nucleic acid amplified in the PCR assay in step (f) and comparing said sequence with the known sequence of the gene of interest to map the intragenic large rearrangement breakpoint identified.

10. A method for predicting a predisposition of a subject to develop a genetic disease or for diagnosing a genetic disease in a subject, comprising detecting an intragenic large rearrangement in at least one gene involved in said genetic disease by
(a) preparing a first collection of labelled nucleic acid molecules from a reference genomic source,
(b) preparing a second collection of labelled nucleic acid molecules from the genomic source to be tested,
(c) contacting said first and said second collection of labelled nucleic acid molecules with a plurality of surface-bound nucleic acids, which comprise:
(i) oligonucleotides ranging in size from 45 to 70 nucleotides in length,
(ii) said oligonucleotides including
sequences representative of locations distributed in said at least one gene involved in said genetic disease, at least 100 kb before the ATG start codon for the 5′ region of said gene and at least 100 kb after the stop codon for the 3′ region of said gene, wherein exonic regions of said gene are fully covered by overlapping oligonucleotides; non-repeated intronic regions of said gene are fully covered by tiling oligonucleotides, said 5′ region of said gene is covered by at least one oligonucleotide every 250-300 bases and said 3′ region of said gene is covered by at least one oligonucleotide every 550-600 bases, and
sequences representative of locations distributed in the genome outside the gene involved in said genetic disease,
(d) determining the hybridization signal intensity of each oligonucleotide representative of a location distributed in the exons and/or introns of the gene involved in said genetic disease by subtracting
the background noise obtained by evaluating the binding of the first and the second collection of nucleic acids to said plurality of surface-bound nucleic acids representative of locations distributed in the genome outside the gene involved in said genetic disease, from
the binding of the first and the second collection of nucleic acids to said plurality of surface-bound nucleic acids representative of locations distributed in the exons and/or introns of the gene involved in said genetic disease,
thereby detecting at least one intragenic large rearrangement in said gene involved in said genetic disease.

11. The method according to claim 10 for predicting a predisposition to cancer in a subject or for diagnosing a cancer in a subject.

12. The method according to claim 10 for predicting a predisposition to cancer in a subject or for diagnosing a cancer in a subject, wherein said at least one gene involved in cancer is selected from the group consisting of BRCA1, BRCA2, BRIP, PALPB1, CHEK2, PTEN, STK11, CDH1, CASP8, FGFR2, MAP3K1, ATM and TP53 genes for ovarian and breast cancers or in the group consisting of MSH2, MLH1, MSH6, MSH3, PMS1, TFGBR2, MLH3, PMS2, MYH, AXIN2 and APC genes for colorectal cancers.

13. The method according to claim 10 for predicting a predisposition to mucoviscidosis in a subject or for diagnosing mucoviscidosis in a subject, wherein said at least one gene of interest is CFTR.

14. Array comprising a plurality of surface-bound oligonucleotides, wherein said oligonucleotides:
(i) range in size from 45 to 70 nucleotides,
(ii) correspond to
sequences representative of locations distributed in the exons and/or introns of the gene of interest, wherein exonic regions of the gene of interest are fully covered by overlapping oligonucleotides and non-repeated intronic regions of the gene of interest are fully covered by tiling oligonucleotides, and
sequences representative of locations distributed in the genome outside the gene of interest.

15. Array according to claim 14, wherein further comprising oligonucleotides including sequences representative of locations distributed at least 100 kb before the ATG start codon for the 5′ region of the gene of interest and at least 100 kb after the stop codon for the 3′ region of the gene of interest, wherein said 5′ region of the gene of interest is covered by at least one oligonucleotide every 250-300 bases and said 3′ region of the gene of interest is covered by at least one oligonucleotide every 550-600 bases.

16. Array according to claim 14, wherein said at least one gene of interest is selected in the group consisting of BRCA1, BRCA2, BRIP, PALPB1, CHEK2, PTEN, STK11, CDH1, CASP8, FGFR2, MAP3K1, ATM, TP53, MSH2, MLH1, MSH6, MSH3, PMS1, TFGBR2, MLH3, PMS2, MYH, AXIN2, APC and CFTR.

17. A method for validating the detection of an intragenic large rearrangement in a gene of interest, comprising
(a) when the intragenic large rearrangement identified is a deletion, designing sens and antisens primers from the non-deleted oligonucleotides surrounding in 5′ and 3′ the sequence deleted,
(a′) when the intragenic large rearrangement identified is a duplication, designing sens and antisens primers from the duplicated oligonucleotides present in 5′ and 3′ of the sequence duplicated,
(b) carrying out a quantitative or semi-quantitative PCR with the primers designed in step (a) or (a′) on nucleic acids from reference genomic source and genomic source to be tested,
(c) determining an amplification of nucleic acids from genomic source to be tested, thereby validating the presence of an intragenic large rearrangement in the gene of interest.


SEQUENCE LISTING (4 sequences)

SEQ ID NO: 1 - DNA, 24 bp, Artificial Sequence
  Description of Artificial Sequence Synthetic primer
catcagatacaccaaaaagacaga

SEQ ID NO: 2 - DNA, 25 bp, Artificial Sequence
  Description of Artificial Sequence Synthetic primer
tatttactcctccaaatgtatcact

SEQ ID NO: 3 - DNA, 43 bp, Artificial Sequence
  Description of Artificial Sequence Synthetic oligonucleotide
ggctgggcgtagtagcttgcacctgtaatcccatcactttggg

SEQ ID NO: 4 - DNA, 35 bp, Artificial Sequence
  Description of Artificial Sequence Synthetic oligonucleotide
atgctggtacagttttcttcctggtgtgatgttgt